(19) United States
(12) Patent Application Publication
(10) Pub. No.: US 2013/0072553 A1
(43) Pub. Date: Mar. 21, 2013

(54) DRUGS, DERIVATIVES AND ANALOGS CONTAINING ADAMANTANE STRUCTURES OF NEW INDICATION APPLICATIONS OF ANTI-TUMOR

(71) Applicant: Lifeng Xu, San Diego, CA (US)

(72) Inventor: Lifeng Xu, San Diego, CA (US)

(21) Appl. No.: 13/376,155

(22) PCT Filed: Dec. 25, 2009
(86) PCT No.: PCT/CN09/76061

§ 371 Date: Jan. 10, 2012

(30) Foreign Application Priority Data
Jun. 11, 2009 (CN) 200910146141.3

(51) Int. Cl. A61K 31/13; C07C 211/19; C07C 271/24; A61K 45/06; C07C 49/513; A61K 31/27; A61K 31/192; A61K 31/122; C07C 211/38; C07C 65/26

(52) U.S. Cl. CPC A61K 31/13; C07C 211/38; C07C 211/19; C07C 271/24; C07C 65/26; C07C 49/513; A61K 31/27; A61K 31/192; A61K 31/122; A61K 45/06
USPC 514/479; 564/459; 564/456; 560/117; 562/467; 568/373; 514/662; 514/659; 514/569; 514/691

(57) ABSTRACT

This invention relates to the drugs, derivatives and analogs containing adamantane structures of new indication applications of anti-tumor. The invention also relates to above compounds to treat tumors and other diseases with FIG. 1. These drugs, derivatives and analogs are generated by the modification of the parent or fragment structures and formation of pharmaceutically acceptable salts, complex salts or prodrug. The drugs, derivatives and analogs as described are administered alone or together with at least one known anti-tumor and immune chemotherapeutic agent including treatment of viral, bacterial and fungal diseases, neurological diseases, endocrine system diseases, and immune system diseases.

### THE FIELD OF INVENTION

This invention relates to the drugs, derivatives and analogs containing adamantane structures of new indication applications of anti-tumor. The invention also relates to above compounds to treat tumors and other diseases.

### BACKGROUND ART

Adamantane drugs, Amantadine (chemical structure see FIG. 1, compound 1), Rimantadine (chemical structure see FIG. 1, compound 2), Memantine (chemical structure see FIG. 1, compound 3), Tromantadine (chemical structure see FIG. 1, compound 4), Adapalene (chemical structure see FIG. 1, compound 5) and Idramantone (chemical structure see FIG. 1, compound 6) have the adamantane structure. They have been used as drugs to treat virus, acne and tuberculosis for many years. Additional new indications and applications of mentioned adamantane structural compounds and drugs have been reported by the patents, including new anti-virus applications, nervous system activity, neuroprotective effect, dermatology applications, respiratory tract infection treatment, tuberculosis treatment. So far the anti-tumor application of mentioned adamantane drugs have not been reported except U.S. Pat. No. 7,250,394, reported that memantine treats glial cell proliferation due to brain cancer, cervical glioma.

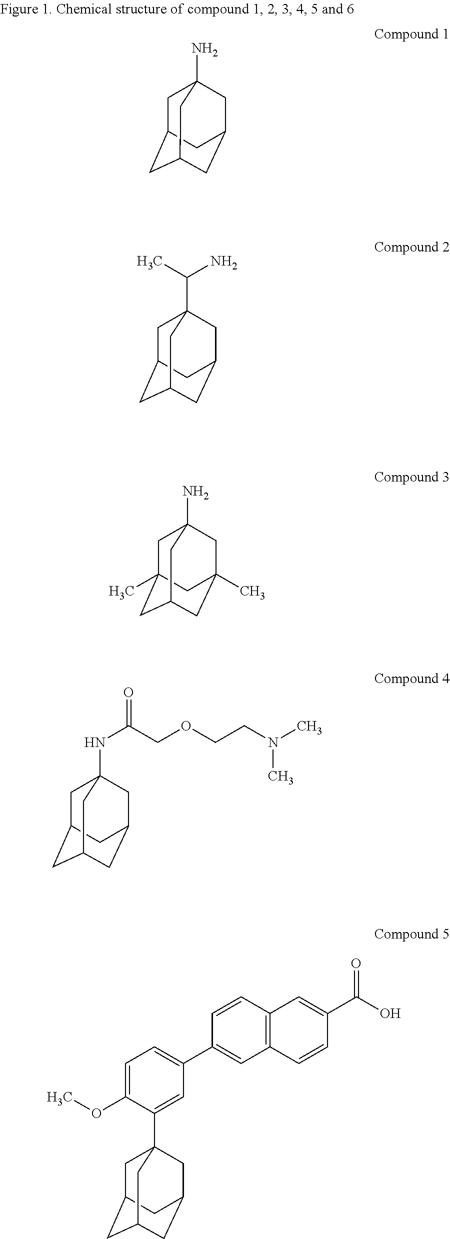

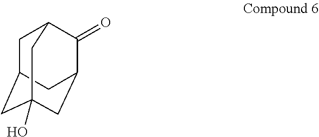 [SMILES: O=C1C2CC3CC1CC(O)(C3)C2]

Amantadine (1-Adamantanamine, 1-Adamantylamine, 1-Aminoadamantane, Amantadina, Aminotricyclo(3,3,1,13,7)decane, Tricyclo(3.3.1.13,7)decane-1-methanamine, Adamantanamine, Adamantylamine, Aminoadamantane, BRN 2204333, EINECS 212-201-2, HSDB 3202, NSC 341865, Pkmerz and Amantadinum), chemical registration No.: 768-94-5, 665-66-7 (Hydrochloride), molecular formula C₁₀H₁₇N, molecular weight 151.25. Amantadine was synthesized by Setter in 1959 (literature 1: The Merck Index 11th Ed 1989. P. 60), Davis first found to have antiviral activity in 1964 (Literature 2: Florey K. “Analytical Profiles of Drug Substances” Vol. 12 P. 2. Academic Press New York 1983). Amantadine was approved as anti-influenza drug by U.S. Pat. No. 5,849,800 reported the treatment of hepatitis C by amantadine as well as the treatment of nerve damage, by European Patent EP 0660707B1.

Rimantadine (1-(1-Adamantyl)ethylamine, Rimantadina, Rimantadinum, Flumadine, Tricyclo-(3,3,1,13,7) decane-1-methanamine, alpha-Methyl-1-adamantanemethylamine, alpha-Methyladamantane methylamine, 1-Adamantanemethylamine, BRN 2715740, HSDB 7438, chemical registry number: 13392-28-4, 1501-84-4 (Hydrochloride)), Molecular formula: C₁₂H₂₁N, molecular weight: 179.3. Varieties activities of rimantadine have been reported, for example, treatment and prevention of influenza by US 20090081249, US 20080045472, U.S. Pat. No. 5,243,030, U.S. Pat. No. 5,824,536, U.S. Pat. No. 7,223,790, U.S. Pat. No. 7,230,028, U.S. Pat. No. 7,157,494, U.S. Pat. No. 7,214,707, U.S. Pat. No. 7,504,434, WO/2009/032605A2, WO/2007/129290A1, WO/2008/087441A2 and WO/2008/022303A2, the treatment of infectious respiratory diseases by US 20030045577, US 20020004046, US 20070092523, US 20060008459, WO/2001/082965A1 and EP 1278545A1, treatment of hepatitis B and hepatitis C by U.S. Pat. No. 6,495,521, US 20050119318, US 20080014173, US 20080311075, WO/1998/019670A2, WO/2008/008776A2 and WO/2006/086381A2, treatment of nerve damage by U.S. Pat. No. 5,614,560, U.S. Pat. No. 5,747,545, U.S. Pat. No. 5,334,618, EP 0660707B1, WO/1992/017168A1 and WO/19941005275A1, the treatment of Parkinson's disease by US 20050245617, the Treatment of glaucoma disease by U.S. Pat. No. 5,922,773, US 20050147584.

Memantine, 1,3-Dimethyl-5-adamantanamine, 1-Amino-3,5-dimethyladamantane, Memantina, 3,5-Dimethyl-1-adamantylamine, 3,5-Dimethyltricyclo(3,3,1,13,7) decan-1-amine, Memantinum, 5-Dimethyl-1-adamantanamine, Tricyclo (3,3,1,13,7) decan-1-amine, DMAA, HSDB 7327, Chemical Registration Numbers: 19982-08-2, 51052-62-1, 41100-52-1 (Hydrochloride), Formula C₁₂H₂₁N, Molecular Weight: 179.3. Varieties activities of Memantine have been reported, for example, treatment of depression, psychiatric disorders, schizophrenia, by US 20060270742, US 20060035888, US 20080282911, US 20080249082, US 20060252788, US20050245460, US 20050288375 and U.S. Pat. No. 7,456,224, the treatment of Alzheimer's disease by US 20080044461, US 60020042, US 20060020042, US 200600240007, US 20050203191 and US 20040092427, the treatment of Parkinson's disease by US 20070026054, the treatment of migraine by U.S. Pat. No. 5,939,425 and U.S. Pat. No. 5,891,885, the treatment of the cancer glioma and cervical glioma by U.S. Pat. No. 7,250,394 and US 20050192322, the treatment of glaucoma disease by US 20070293558, U.S. Pat. No. 7,494,983, U.S. Pat. No. 5,922,773 and US 20010047012.

Tromantadine (2-(2-(Dimethylamino) ethoxy)-N-tricyclo (3,3,1,13,7) dec-1-ylacetamide, Acetamide, 2-(2-(dimethylamino) ethoxy)-N-tricyclo (3.3.1.13,7) dec-1-yl-, EINECS 258-770-O, N-(Tricyclo(3,3,1,13,7) decyl)-2-(2-dimethylaminoethoxy)acetamid, N-1-Adamantyl-2((2-dimethylamino)ethoxy)acetamide, Tromantadina, Tromantadinum, UNII-H191JFG8WA, 2-(2-(Dimethylamino) ethoxy)N-tricyclo(3,3,1,13,7) dec-1-ylacetamide, N-1-Adamantyl-2-(2-(dimethylamino) ethoxy)acetamide, chemical registration number, 53783-83-8, 41544-24-5 mono-hydrochloride, Molecular formula: C₁₆H₂₈N₂O₂, molecular weight: 280.41. Tromantadine showed the treatment of Parkinson's disease by U.S. Pat. No. 4,277,437, U.S. Pat. No. 6,057,364 and U.S. Pat. No. 6,384,083.

Adapalene (6-(3-(1-Adamantyl)-4-methoxyphenyl)-2-naphthoic acid, Adapaleno, Adapalenum, Differin, 2-Naphthalenecarboxylic acid, 6-(4-methoxy-3-tricyclo (3,3,1,13,7) dec-1-ylphenyl), Registration No. 106685-40-9, molecular formula C₂₈H₂₈O₃, molecular weight: 412.52.

Adapalene showed the treatment of skin diseases and acne treatment by US 20080064755, US 20080033047, US 20080161273, US 20060128808, US 20020155180 and US 20030147977.

Idramantone (Kemantane, 5-Hydroxyadamantan-2-one, 5-Hydroxy-2-adamantanone, Adamantan-4-on-1-ol, Opreal-254797, STOCK1S-56048, CHEBI: 48581, CPD-744), Molecular Formula: C₁₀H₁₄O₂, molecular weight: 166.22, Chemical Registration Numbers: 20098-14-0.

Idramantone showed the treatment of tuberculosis disease (literature 3: Probl Tuberk 1991; (6):23-6. Russian), virus disease (Literature 4: Dokl Biochem Biophys, 2001 May-June, 378: 210-3); chronic bronchitis (literature 5: Lik Sprava. 1992 April; (4):73-6. Russian; literature 6: Zh Mikrobiol Epidemiol Immunobiol. 1991 September; (9):66-7. Russian)

### SUMMARY OF THE INVENTION

The purpose of the present invention is to provide the drugs, derivatives and analogs containing adamantane structures of new indication applications of anti-tumor. The invention also relates to above compounds to treat tumors and other diseases and provide pharmacological activity and experimental methods.

These drugs are Amantadine, Rimantadine, Memantine, Tromantadine, Adapalene, and Idramantone and their chemical structures are listed in FIG. 1, compound 1, compound 2, compound 3, compound 4, compound 5 and compound 6.

The drugs, derivatives and analogs as described are compound 1, compound 2, compound 3, compound 4, compound 5 and compound 6 or are modified to form the derivatives and analogs containing adamantane structures, which have the same or similar structural nucleus and the similar biological activities.

The drugs, derivatives and analogs as described are administered alone or together with at least one known anti-tumor and immune chemotherapeutic agent in the dose of 0.001 mg/kg-2.50 g/kg (intravenous, intraperitoneal or oral administration); a cancer is selected from the group consisting of lung cancer, stomach cancer, colon cancer, small cell lung cancer, thyroid cancer, esophageal cancer, pancreatic cancer, endometrial cancer, adrenal cancer, head and neck cancer, osteogenic sarcoma, breast cancer, ovarian cancer, Vail Williams tumors, cervical tumors, testicular cancer, genitourinary cancer, skin cancer, renal cell cancer, bladder cancer, primary brain cancer, prostate cancer, soft tissue sarcoma, neuroblastoma, rhabdomyosarcoma, Kaposi's sarcoma, malignant melanoma, malignant pancreatic islet tumors, non-Hodgkin's lymphoma, malignant melanoma, multiple myeloma, neuroblastoma, malignant carcinoid cancer, choriocarcinoma, acute and chronic lymphocytic leukemia, primary macroglobulinemia, chronic myeloid leukemia, chronic lymphocytic leukemia, acute myeloid leukemia, hairy cell leukemia, mycosis fungoides, malignant hypercalcemia, cervical hyperplasia, Hodgkin's disease and other and related tumors or diseases.

The drugs, derivatives and analogs as described are administered alone or together with at least one known anti-tumor and immune chemotherapeutic agent, antiviral agents, their salts or prodrugs selected from the group consisting of cyclophosphamide, vincristine, busulfan, vinblastine, cisplatin, carboplatin, mitomycin C, doxorubicin, colchicine, etoposide, paclitaxel, docetaxel, camptothecin, topotecan, arsenic trioxide, 5-ammocytidine, 5-fluorouracil, methotrexate, 5-fluoro-2-deoxy-uridine, hydroxyurea, thioguanine, melphalan, chlorambucil, ifosfamide, mitoguazone, epirubicin, aclarubicin, bleomycin, mitoxantrone, fludarabine, octreotide, tamoxifen, doxazosin, terazosin, tamsulosin, fluorine pyridinoline, lovastatin, simvastatin, pravastatin, fluvastatin, atorvastatin, cerivastatin, amprenavir, abacavir, ritonavir, saquinavir, rofecoxib, alanosine, retinal, tretinoin tocoferil, 13-cis-retinoic acid, 9-cis-retinoic acid, difluoromethylornithine, fenretinide, N-4-hydroxyphenyl retinode, genistein, ara-C, CB-64D, CB-184, ILX23-7553, lactacystin, MG-132, PS-341, Glcevec, ZD1839 (IRessa), SH268, Herceptin, Rituxan, Gamcitabine, ABT-378, AG1776, BMS-232, 632, CEP2563, SU6668, EMD121974, R115777, SCH66336, L-778, 123, BAL9611, TAN-1813, UCN-01, roscovitine, olonoucine, valecoxib.

In addition, the drugs, derivatives and analogs as described: in the treatment of cancer and other diseases associated with apoptosis related diseases and applications, including virus infection and neurological disorders application, pharmacologically acceptable salt and prodrug applications, and other known anti-virus and anti-nervous system diseases or drugs combined applications. The administration may be by oral route, parenteral, subcutaneous, intravenous, intramuscular, intra-peritoneal, transdermal, buccal, intrathecal, intracranial, intranasal or topical routes.

The invention has the following beneficial effects: In order to raise the polarity of the drugs, derivatives and analogs, the salts were prepared to obtain injection with good water solubility, improved bioavailability and better the anti-tumor activity by the injection process method.

### BRIEF DESCRIPTION OF THE DRAWINGS

FIG. 2 shows five kinds of test substance and positive control cyclophosphamide (CTX) inhibition of colon cancer (HT-29) growth in vitro (Example 1, 2, 3, 4, 5, 6, 7, 8, 9, 10, 11, 12, 13, 14, 15, 16 and 17).

FIG. 3 shows the six kinds of test substance and positive control cyclophosphamide (CTX) inhibition of pancreatic cancer cells (Panc-1) growth in vitro (Example 1, 2, 3, 4, 5, 6, 7, 8, 9, 10, 11, 12, 13, 14, 15, 16 and 17).

FIG. 4 shows the five kinds of test substance and positive control cyclophosphamide (CTX) inhibition of lung cancer cells (NCI-H460) growth in vitro ((Example 1, 2, 3, 4, 5, 6, 7, 8, 9, 10, 11, 12, 13, 14, 15, 16 and 17).

FIG. 5 shows the five kinds of test substance and positive control cyclophosphamide (CTX) inhibited breast cancer cells (MCF7) growth in vitro (Example 1, 2, 3, 4, 5, 6, 7, 8, 9, 10, 11, 12, 13, 14, 15, 16 and 17).

### METHODS

### 1. Source of Test Compounds

Amantadine was purchased from Xin Xiang Rong Pharmaceutical Co., Ltd. Shijiazhuang City, China; Rimantadine was purchased from Dongtai Pharmaceutical Co., Ltd., Henany, China; Memantine was purchased from Hiromi Pharmaceutical Co., Ltd. Shenyangy, China; Tromantadine was purchased from the Chiral Drugs Research Institute Co., Ltd., Chengdu City, China; Adapalene was purchased from Tongfeng Pharmaceutical Co., Ltd., Zhejiang, China; Idramantone was purchased from Yuancheng Science and Technology Co., Ltd. Wuhan City, China.

### 2. Identification of Test Compounds

Test compounds, Amantadine, Rimantadine, Memantine, Tromantadine, Adapalene and Idramantone identified by infrared spectroscopy, NMR spectroscopy, Mass spectrometry and purity analysis are consistent with literatures (literature 7: USP 30; literature 8: CP 2005; literature 9: EP5; Literature 10: K. Gerzon et al, J. Med. Chem. 6, 760 (1963); literature 11: Ger. Patent 1, 941, 218, 1971; literature 12: Idramantone Probl Tuberk. 1991, (6):23-6. Russian.) (details see Examples).

### 3. Salt Preparation of Test Compounds

Pharmaceutically acceptable salts can be made by acid or base, such as hydrochloric acid, fumaric acid, maleic acid, succinic acid, acetic acid, citric acid, tartaric acid, carbonic acid, phosphoric acid, oxalic acid, etc or sodium carbonate, sodium hydride, potassium hydroxide, ammonium hydroxide, etc.

The compound containing amino substituents was quantitatively dissolved in methanol, acetone, tetrahydrogen furan or 1,4-dioxane to form clear solution. Dry hydrogen chloride gas or other acid was led into the solution until completely the solution was saturated under the ice water bath and stirring. Then to maintain the salt reaction 1-2 hours until the reaction was completed. The solvent was evaporated under vacuum to obtain the salt.

The compound containing acidic substituents was quantitatively dissolved in methanol, acetone, tetrahydrogen furan or 1,4-dioxane to form clear solution. The equivalent of base solution (2N), was led into the solution until the reaction is complete, under the ice water bath and stirring. The solvent was evaporated under vacuum to obtain the salt. The drugs, derivatives and analogs of this invention are selected from the exemplified examples or pharmaceutically acceptable salts formed by the reaction of acid and base, such as hydrochloric acid, fumaric acid, maleic acid, succinic acid, acetic acid, citric acid, tartaric acid, carbonic acid, phosphoric acid, oxalic acid, sodium carbonate, sodium hydride, potassium hydroxide, ammonium hydroxide, etc. Prodrug of this invention can be made to increase its water solubility and bioavailability. Pharmaceutical compounds of this invention can be administered by oral, parenteral, subcutaneous, intravenous, intramuscular, intraperitoneal, transdermal, buccal, intrathecal, intracranial, intranasal administration or local channels. Dose range is 0.001 mg/kg-2.50 g/kg (intravenous, intraperitoneal or oral administration, etc.).

The following examples are illustrative, but not limiting, of the method and composition of the present invention.

### Example 1

### Structural Identification of Amantadine

IR (KBr, cm⁻¹) 3437, 3183, 3033, 2925, 2853, 1599, 1493, 1477, 1453, 1376, 1365, 1312, 1085; 1H NMR (CDCl₃) δ 8.19 (b, 2H), 2.08 (m, 4H), 1.82 (m, 3H), 1.70 (m, 4H), 1.57 (m, 4H).

### Example 2

### Structural Identification of Rimantadine

IR (KBr, cm⁻¹) 3438, 3031, 2904, 2850, 1602, 1505, 1450, 1392, 1374, 1341, 1263, 1208, 1190, 1077, 984; 1H NMR (DMSO-d₆) δ 7.94 (b, 2H), 3.40 (m, 2H), 2.76 (m, 1H), 1.67 (m, 4H), 1.57 (m, 8H), 1.49 (m, 4H).

### Example 3

### Structural Identification of Memantine

IR (KBr, cm⁻¹) 3419, 2943, 2900, 2861, 2707, 2613, 2055, 1615, 1603, 1513, 1456, 1364, 1357, 1344, 1322; 1H NMR (DMSO-d₆) δ 8.24 (b, 2H), 2.15 (m, 1H), 1.65 (m, 2H), 1.45 (m, 4H), 1.29 (m, 4H), 1.06 (m, 2H), 0.85 (s, 6H).

### Example 4

### Structural Identification of Tromantadine

1H NMR (CDCl₃) δ 6.78 (s, 1H), 3.83 (s, 2H), 3.75 (m, 2H), 2.51 (m, 2H), 2.28 (s, 6H), 2.07 (s, 3H), 2.02 (m, 6H), 1.68 (m, 6H); MS/e: 281 (M+1).

### Example 5

### Structural Identification of Adapalene

IR (KBr, cm⁻¹) 3446, 2902, 2849, 2656, 1689, 1629, 1601, 1476, 1303, 1236, 1101, 805, 704; 1H NMR (DMSO-d₆) δ 8.61 (s, 1H), 8.22 (s, 1H), 8.16 (d, J=9.0 Hz, 1H), 8.08 (d, J=9.0 Hz, 1H), 7.99 (d, J=8.40 Hz, 1H), 7.90 (d, J=8.40 Hz, 1H), 7.66 (d, J=8.40 Hz, 1H), 7.58 (d, J=8.40 Hz, 1H), 7.12 (d, J=8.40 Hz, 1H), 3.87 (s, 3H), 2.14 (m, 6H), 2.07 (m, 3H), 1.76 (m, 6H).

### Example 6

### Structural Identification of Idramantone

IR (KBr, cm⁻¹) 3410, 2920, 2810, 1721, 1440, 1330, 1241, 1060, 881; MS/e: 167 (M+1), 148, 119.

### Examples of Anti-Tumor Agents

### Example 7

Anti-tumor agent 1: compound 1. 5.0 g (the compound described in Example 1), ethanol 600 ml, 1,2-propanediol 600 ml and Tween 80 100 ml were dissolved and the injection water was added up to total volume of 5000 ml. The solution was filtered with 0.22 μm membrane filter and sterilized for 30 min at 100° C. to obtain 1000 preparation of injection 5 mg/5 ml.

### Example 8

Anti-tumor agent 2: compound 2. 8.0 g (the compound described in Example 2), dimethyl sulfoxide (DMSO) 50 ml, 1,2-propanediol 500 ml and Tween 80 100 ml were dissolved and the injection water was added up to total volume of 5000 ml. The solution was filtered with 0.22 μm membrane filter and sterilized for 30 min at 100° C. to obtain 1000 preparation of injection 8 mg/5 ml.

### Example 9

Anti-tumor agent 3: compound 3. 8.0 g (the compound described in Example 3), dimethyl sulfoxide (DMSO) 50 ml, 1,2-propanediol 500 ml and Tween 80 100 ml were dissolved and the injection water was added up to total volume of 5000 ml. The solution was filtered with 0.22 μm membrane filter and sterilized for 30 min at 100° C. to obtain 1000 ml preparation of injection 8 mg/5 ml.

### Example 10

Anti-tumor agent 4: compound 4. 8.0 g (the compound described in Example 4), dimethyl sulfoxide (DMSO) 50 ml, 1,2-propanediol 500 ml and Tween 80 100 ml were dissolved and the injection water was added up to total volume of 5000 ml. The solution was filtered with 0.22 μm membrane filter and sterilized for 30 min at 100° C. to obtain 1000 preparation of injection 8 mg/5 ml.

### Example 11

Anti-tumor agent 5: compound 5. 8.0 g (the compound described in Example 5), dimethyl sulfoxide (DMSO) 50 ml, 1,2-propanediol 500 ml and Tween 80 100 ml were dissolved and the injection water was added up to total volume of 5000 ml. The solution was filtered with 0.22 μm membrane filter and sterilized for 30 min at 100° C. to obtain 1000 preparation of injection 8 mg/5 ml.

### Example 12

Anti-tumor agent 6: compound 6. 4.0 g (the compound described in Example 6), dimethyl sulfoxide (DMSO) 50 ml, 1,2-propanediol 500 ml and Tween 80 100 ml were dissolved and the injection water was added up to total volume of 5000 ml. The solution was filtered with 0.22 μm membrane filter and sterilized for 30 min at 100° C. to obtain 1000 preparation of injection 8 mg/5 ml.

### Examples of Anti-Tumor In Vitro Experiments

### Example 13

### In Vitro Anti-Cancer Cell Experiment

Cell lines: Human pancreatic cancer cell line Panc-1, human colorectal cancer cell line HT29, human lung cancer cell line NCI-H460 and human breast cancer cell line MCF7; the medium: s DMEM (Gibco BRL), containing 10% fetal calf serum (Gibco BRL) and 2 mM L-glutamine (Gibco BRL).

(2) Test Samples: Example Compound 1, 2, 3, 4, 5 and 6.

The samples were dissolved in dimethyl sulfoxide (DMSO, Sigma, United States) and medium was added to the final concentration of 0.5%. cyclophosphamide was as positive control (CTX, purity>96%).

Method: cells were digested with trypsin and dispersed into single cells in the medium containing penicillin (25 U/ml) and streptomycin (25 μg/ml). The cells were seeded in 96-well culture plates (Corning Incorporated), at 37° C., with 5% CO₂ present for 24 hours. The culture medium was removed, 1-100 μm test compounds were added, cultured for 48 hours. Culture medium was removed and thiophene Wow blue (MTT, USA Sigma products) was added. The result was assayed by SK601-based microplate reader (Japan Seikagaku company's products), 570 nm/630 nm optical density (OD). Cell viability determined by the formula: (Experimental group OD/control OD)×100%; CDDP and the positive control subjects treated in the same material simultaneously.

Inhibition of colorectal cancer: as shown in FIG. 1, and table 1 five test compounds showed anti-proliferative effect on HT29. Example compounds 8 showed close values to CTX of anti-proliferate on HT29, IC₅₀ (the compound concentration producing 50% inhibition of colony formation) values, and 95% confidence limit of 3.92 (1.99-5.46) μg/ml, CTX of the IC₅₀ 95% confidence limit of 2.26 (2.08-4.89) μg/ml; anti-proliferative effect of compound 7, 9 and 10 is relatively weak, IC₅₀ and 95% confidence limits were 18.81 (15.7-21.38) μg/ml, 13.13 (9.54-15.31) μg/ml and 10.21 (8.01-11.74) μg/ml. Anti-proliferative effect of compound 5 is the strongest, IC₅₀ and 95% confidence limit of 0.85 (0.61-1.32) μg/ml by twice experiment with good reproducibility.

### Example 14

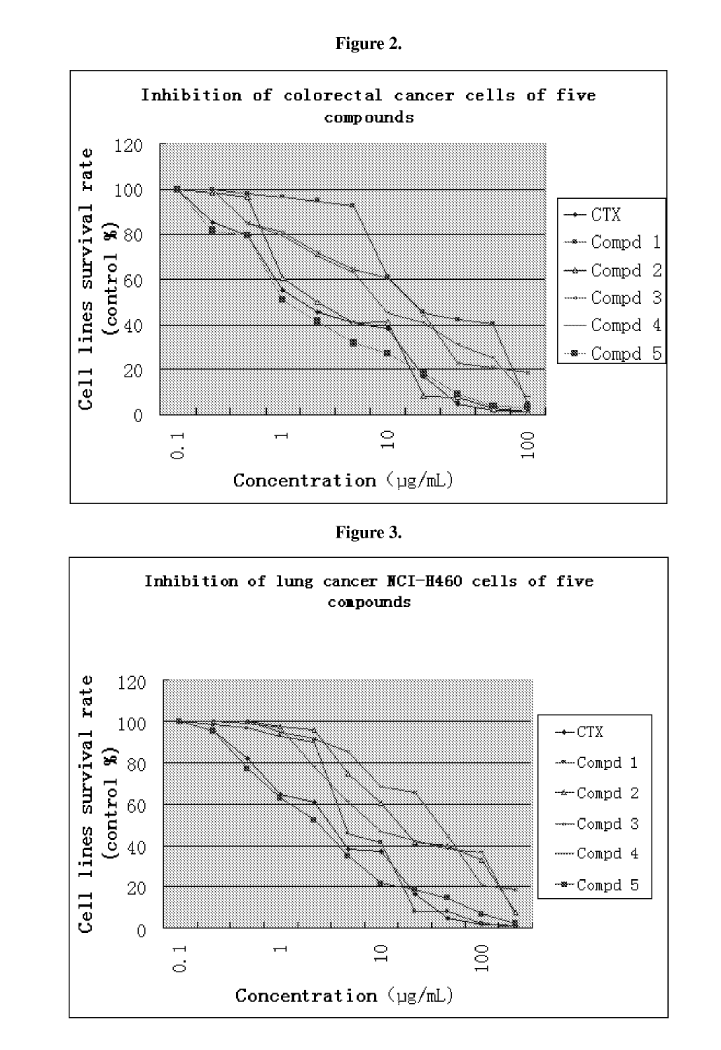

Inhibition of pancreatic cancer: as shown in FIG. 2, and table 2 six test compounds showed anti-proliferative effect on Panc-1. Example compounds 8 and 11 showed anti-proliferative effect on Panc-1 at IC₅₀ (the compound concentration producing 50% inhibition of colony formation) values 2.42 (1.25-3.51) μg/ml and 2.17 (0.89-3.45) μg/ml, IC₅₀ (P<0.05), compound 7, 9, 10 and 12 of Panc-1 half inhibitory concentration (IC₅₀) and 95% confidence limits were 51.32 (48.95-55.51) μg/ml, 52.97 (50.20-54.41) μg/ml, 10.01 (7.24-12.91) μg/ml and 53.45 (45.32-58.67) μg/ml. Compound 11 a strong anti-proliferative effect, better than CTX IC₅₀ and 95% confidence limit of 2.23 (1.81-2.46) μg/ml by twice experiment with good reproducibility.

### Example 15

Inhibitory effect on lung cancer NCI-H460 cells: as shown in FIG. 3. Positive control IC₅₀ of CTX and 95% confidence limit of 4.30 (3.04-5.13) μg/ml. Test compound 7 and 10 of the IC₅₀ and 95% confidence limits were 5.18 (4.42-7.03) and 8.52 (6.66-9.14). IC₅₀ value of test compound 8, 9 and 11 are 22.01 (20.42-25.03), 60.52 (58.66-62.14) and 2.81 (1.99-3.23) with 95% confidence limits. Compound 11 and compound 7 is more sensitive to NCI-H460 cells, by twice experiment with good reproducibility.

### Example 16

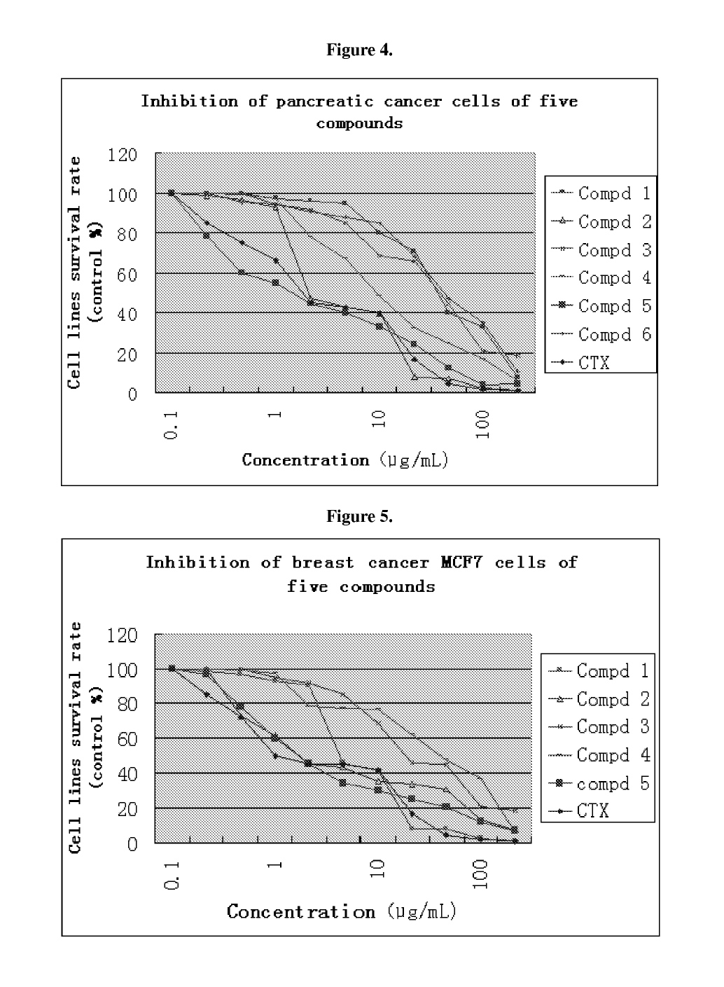

Inhibitory effect on breast cancer MCF7 cells: as shown in FIG. 4, test compound 1, 2, 3, 4 and 5 of the IC₅₀ were respectively 6.18 (3.42-8.03), 2.73 (1.21-4.02), 34.33 (30.54-40.78), 67.45 (58.89-79.51) and 2.94 (0.78-4.06) with 95% confidence limits. Positive control of IC₅₀ of CTX and 95% confidence limit of 0.92 (0.03-2.02) μg/ml. Compound 11 is more sensitive to NCI-H460 cells, IC₅₀ (P<0.05). In this experiment twice, good reproducibility. Inhibitory effect on colorectal cancer cell lines HT-29, pancreatic cancer Panc-1, lung cancer NCI-H460 and breast MCF7 of test compound 1, 2, 3, 4 and 5 with cyclophosphamide as a control experiment showed that all compounds are sensitive to colorectal cancer, pancreatic cancer and breast cancer cells. Compound 5 and 2 showed higher inhibition activity on colorectal cancer and pancreatic cancer.

Cancer cells in vitro results in Table 1.

TABLE 1
Cancer cells in vitro results
Six compounds IC₅₀ (μM)

| | Colorectal | Pancreatic | Lung | Breast |
|---|---|---|---|---|
| | cancer | cancer | cancer | cancer |
| IC50 | HT-29 | Panc-1 | NCI-H460 | MCF7 |
| Compound 1 | 18.81 | 51.32 | 5.18 | 6.18 |
| Compound 2 | 3.92 | 2.42 | 22.01 | 2.73 |
| Compound 3 | 13.13 | 52.97 | 60.52 | 34.33 |
| Compound 4 | 10.21 | 10.01 | 8.52 | 67.45 |
| Compound 5 | 0.85 | 2.17 | 2.81 | 2.94 |
| Compound 6 | | 53.45 | | |
| CTX | 2.26 | 2.23 | 4.30 | 0.92 |

### Example of Anti-Tumor Experiments In Vivo

### Example 17

### (1) Materials

Test samples: example compounds 1, 2, 3, 4, 5 and 6

Test animals: Kunming kinds of healthy mice (19-21 g), 10 mice (5 male and 5 female)/group, from Beijing Institute of Military Medical Sciences Animal Center.

Tumor strains: mice sarcoma S180 for ascites passaged from Beijing Academy of Military Medical Institute of Pharmacology.

### (2) Methods

Xenografts cultured S180 tumor cells were implanted subcutaneously into the flank region of mice and tumors were allowed to grow to the desired average size of 100 mg. The mice were randomized into control and treatment groups with 10 mice per group. The control group was injected with the vehicle used to dissolve the drug. Other groups received the test compounds (example compound 1, 2, 3, 4, 5 and 6, respectively 25 mg/kg, 40 mg/kg, 12.5 mg/kg, 15 mg/kg and 15 mg/kg), and positive group, cyclophosphamide (CTX) and 5-fluorouracil (5-FU) at the dose 15 mg/kg. Injections were I.V. via the tailvein. Tumor measurements were taken every other day 20% tumor growth inhibition which was not statistically significant.

(3) Results

The in vivo experimental data showed anti-tumor efficacy of example compound 1, 2, 3, 4, 5 and 6 are statistically significant (Table 2).

TABLE 2
inhibition of the sarcoma growth S180 ( X̅ ± SD, n = 16)

| | The | | | | | |
|---|---|---|---|---|---|---|
| | route | body Weight/g | | Tunor | Inhibition | |
| Group | of ad. | Before ad. | After ad. | weight (g) | rate (%) | P < |
| Control | — | 23.87 ± 1.70 | 27.05 ± 3.98 | 2.41 ± 1.22 | — | — |
| CTX | iv | 22.45 ± 0.79 | 28.22 ± 2.46 | 1.04 ± 0.37 | 38.40 | 0.01* |
| 5-FU | iv | 21.61 ± 1.56 | 29.16 ± 2.50 | 1.64 ± 0.50 | 33 | 0.05* |
| Compound 1 | iv | 21.41 ± 2.52 | 27.93 ± 3.37 | 1.27 ± 0.50 | 31.35 | 0.05* |
| Compound 2 | iv | 19.35 ± 0.95 | 26.02 ± 3.69 | 1.06 ± 0.51 | 43.67 | 0.01* |
| Compound 3 | iv | 21.31 ± 1.53 | 28.98 ± 1.74 | 1.10 ± 0.77 | 42.7 | 0.05* |
| Compound 4 | iv | 21.03 ± 0.29 | 28.64 ± 4.13 | 1.23 ± 0.36 | 34.31 | 0.05* |
| Compound 5 | iv | 20.27 ± 1.80 | 20.80 ± 3.61 | 0.81 ± 0.42 | 56.22 | 0.01** |
| Compound 6 | iv | 19.92 ± 1.47 | 26.30 ± 3.62 | 1.70 ± 0.59 | 29.02 | 0.05* |

The route of ad.: The route of administration;
Before ad.: before administration;
After ad.: before administration
P < 0.05 compared with Control group significantly different;
**p < 0.01 compared with the Control group was a significant difference

The test group inoculated tumor cells 5180 in mice armpit, administration and observation of seven days. The data showed that inhibition rate of compound 2, compound 3, compound 4 and compound 5 were better than the positive control group, 5-FU by measuring the tumor weight in mice armpit approach. The inhibition of compound 2, compound 3 and compound 5 were significantly better than the positive control group of cyclophosphamide. The data showed that inhibition rate of compound 2 (40 mg/kg), compound 3 (12.5 mg/kg) and compound 5 (50 mg/kg) were more than 40%.

This invention relates to the drugs, derivatives and analogs containing adamantane structures of new indication applications of anti-tumor.

The invention also relates to above compounds to treat tumors and other diseases. This invention relates to the drugs, derivatives and analogs containing adamantane structures of new indication applications of anti-tumor including: the preparation of anti-tumor activity and the application as anticancer drugs, active ingredients for the chemical structure of compound 1, 2, 3, 4, 5 and 6, its derivatives and analogues compounds as described in FIG. 1, are administered alone or together with at least one known anti-tumor and immune chemotherapeutic agent in the dose of 0.001 mg/kg-2.50 g/kg (intravenous, intraperitoneal or oral administration); a cancer is selected from the group consisting of lung cancer, stomach cancer, colon cancer, small cell lung cancer, thyroid cancer, esophageal cancer, pancreatic cancer, endometrial cancer, adrenal cancer, head and neck cancer, Osteogenic sarcoma, breast cancer, ovarian cancer, Vail Williams tumors, cervical tumors, testicular cancer, genitourinary cancer, skin cancer, renal cell cancer, bladder cancer, brain cancer, prostate cancer, soft tissue sarcoma, neuroblastoma, rhabdomyosarcoma, Kaposi's sarcoma, malignant melanoma, malignant ancreatic islet tumors, non-Hodgkin's lymphoma, malignant melanoma, multiple myeloma, neuroblastoma, malignant carcinoid cancer, choriocarcinoma, acute and chronic lymphocytic leukemia, primary macroglobulinemia, chronic myeloid leukemia, chronic lymphocytic leukemia, acute myeloid leukemia, hairy cell leukemia, mycosis fungoides, malignant hypercalcemia, cervical hyperplasia, Hodgkin's disease and other and related tumors or disease.

The drugs, derivatives and analogs as described are administered alone or together with at least one known anti-tumor and immune chemotherapeutic agent, antiviral agents, their salts or prodrugs selected from the group consisting of cyclophosphamide, vincristine, busulfan, vinblastine, cisplatin, carboplatin, mitomycin C, doxorubicin, colchicine, etoposide, paclitaxel, docetaxel, camptothecin, topotecan, arsenic trioxide, 5-ammocytidine, 5-fluorouracil, methotrexate, 5-fluoro-2-deoxy-uridine, hydroxyurea, thioguanine, melphalan, chlorambucil, ifosfamide, mitoguazone, epirubicin, aclarubicin, bleomycin, mitoxantrone, fludarabine, octreotide, tamoxifen, doxazosin, terazosin, tamsulosin, fluorine pyridinoline, lovastatin, simvastatin, pravastatin, fluvastatin, atorvastatin, cerivastatin, amprenavir, abacavir, ritonavir, saquinavir, rofecoxib, Alanosine, retinal, tretinoin tocoferil, 13-cis-retinoic acid, 9-cis-retinoic acid, difluoromethylornithine, fenretinide, N-4-hydroxyphenyl retinode, genistein, ara-C, CB-64D, CB-184, ILX23-7553, lactacystin, MG-132, PS-341, Glcevec, ZD1839 (IRessa), SH268, Herceptin, Rituxan, Gamcitabine, ABT-378, AG1776, BMS-232, 632, CEP2563, SU6668, EMD121974, R115777, SCH66336, L-778, 123, BAL9611, TAN-1813, UCN-01, roscovitine, olonoucine, valecoxib. In addition, the drugs, derivatives and analogs as described: in the treatment of cancer and otherdiseases associated with apoptosis-related diseases and applications, including virus infection and neurological disorders application, pharmacologically acceptable salt and prodrug applications, and other known anti-virus and anti-nervous system diseases or drugs combined applications.

The administration may be by oral route, parenteral, subcutaneous, intravenous, intramuscular, intraperitoneal, transdermal, buccal, intrathecal, intracranial, intranasal or topical routes.

1. Six drugs, derivatives and analogs containing adamantane structures, the chemical structure formula shown in FIG. 1, compound 1, compound 2, compound 3, compound 4, compound 5 and compound 6: 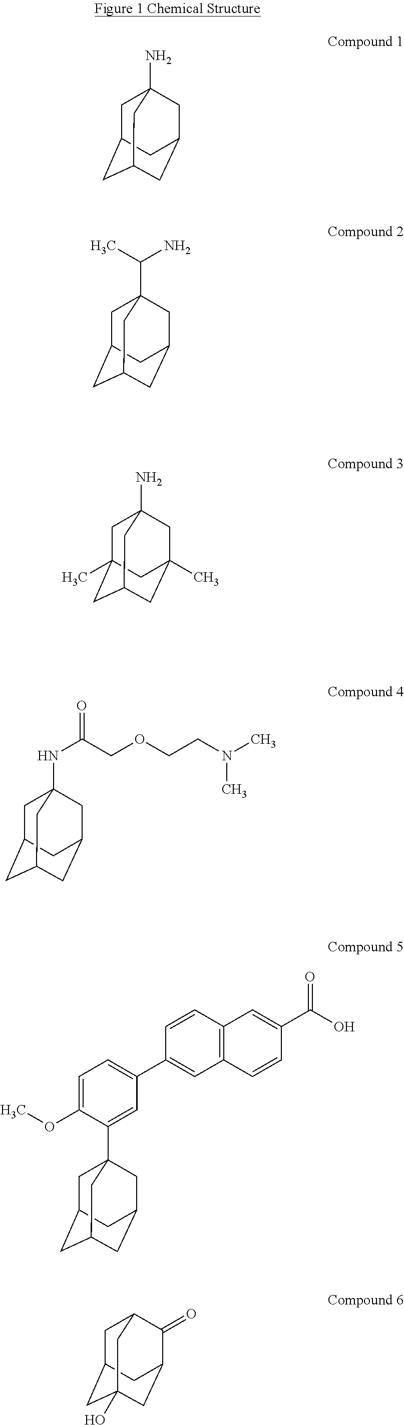
Compound 1 Amantadine, Compound 2 Rimantadine, Compound 3 Memantine, Compound 4 Tromantadine, Compound 5 Adapalene and Compound 6 Idramantone.

2. The drugs, derivatives and analogs according to claim 1, wherein:
compound 1, compound 2, compound 3, compound 4, compound 5 and compound 6, derivatives and analogs are generated by the modification of the parent or fragment structures.

3. The drugs, derivatives and analogs according to claim 2, wherein: the derivatives and analogs prepared by direct synthesis or structural modification of the drugs as described compound 1, compound 2, compound 3, compound 4, compound 5 and compound 6 are relevance to the drugs as described compound 1, compound 2, compound 3, compound 4, compound 5 and compound 6 in chemical structures and pharmaceutical activities.

4. The drugs, derivatives and analogs according to claim 3, wherein:
The drugs, derivatives and analogs are selected from the exemplified examples or pharmaceutically acceptable salts formed by organic acid, inorganic acid, organic base, inorganic base, prodrug or complex salts.

5. The drugs, derivatives and analogs according to claims 1, 2, 3 and 4 wherein:
This invention relates to the drugs, derivatives and analogs containing adamantane structures of new indication applications of anti-tumor including: the preparation of anti-tumor activity and the application as anticancer drugs, active ingredients for the chemical structure of compound 1, compound 2, compound 3, compound 4, compound 5 and compound 6, its derivatives and analogues compounds are administered alone or together with at least one known anti-tumor and immune chemotherapeutic agent in the dose of 0.001 mg/kg-2.50 g/kg (intravenous, intraperitoneal or oral administration); a cancer is selected from the group consisting of lung cancer, stomach cancer, colon cancer, small cell lung cancer, thyroid cancer, esophageal cancer, pancreatic cancer, endometrial cancer, adrenal cancer, head and neck cancer, Osteogenic sarcoma, breast cancer, ovarian cancer, Vail Williams tumors, cervical tumors, testicular cancer, genitourinary cancer, skin cancer, renal cell cancer, bladder cancer, primary brain cancer, prostate cancer, soft tissue sarcoma, neuroblastoma, rhabdomyosarcoma, Kaposi's sarcoma, malignant melanoma, malignant pancreatic islet tumors, non-Hodgkin's lymphoma, malignant melanoma, multiple myeloma, neuroblastoma, malignant carcinoid cancer, choriocarcinoma, acute and chronic lymphocytic leukemia, primary macroglobulinemia, chronic myeloid leukemia, chronic lymphocytic leukemia, acute myeloid leukemia, hairy cell leukemia, mycosis fungoides, malignant hypercalcemia, cervical hyperplasia, Hodgkin's disease and other and related tumors or disease.

6. The drugs, derivatives and analogs according to claim 5 wherein:
The drugs, derivatives and analogs as described are administered alone or together with at least one known anti-tumor and immune chemotherapeutic agent, antiviral agents, their salts or prodrugs selected from the group consisting of cyclophosphamide, vincristine, busulfan, vinblastine, cisplatin, carboplatin, mitomycin C, doxorubicin, colchicine, etoposide, paclitaxel, docetaxel, camptothecin, topotecan, arsenic trioxide, 5-ammocytidine, 5-fluorouracil, methotrexate, 5-fluoro-2-deoxyuridine, hydroxyurea, thioguanine, melphalan, chlorambucil, ifosfamide, mitoguazone, epirubicin, aclarubicin, bleomycin, mitoxantrone, fludarabine, octreotide, tamoxifen, doxazosin, terazosin, tamsulosin, fluorine pyridinoline, lovastatin, simvastatin, pravastatin, fluvastatin, atorvastatin, cerivastatin, amprenavir, abacavir, ritonavir, saquinavir, rofecoxib, Alanosine, retinal, tretinoin tocoferil, 13-cis-retinoic acid, 9-cis-retinoic acid, difluoromethylornithine, fenretinide, N-4-hydroxyphenyl retinode, genistein, ara-C, CB-64D, CB-184, ILX23-7553, lactacystin, MG-132, PS-341, Glcevec, ZD1839 (IRessa), SH268, Herceptin, Rituxan, Gamcitabine, ABT-378, AG1776, BMS-232, 632, CEP2563, SU6668, EMD121974, R115777, SCH66336, L-778, 123, BAL9611, TAN-1813, UCN-01, roscovitine, olonoucine, valecoxib.

7. The drugs, derivatives and analogs according to claims 5 and 6 wherein:
the drugs, derivatives and analogs as described in the treatment of tumor-related disease, cancer or other diseases associated with the apoptosis-related diseases and applications, including viral diseases and low immunity, broad-spectrum bacterial, fungal and bacterial diseases, fungal diseases, bacterial infections, fungal infections diseases, nervous system disease. The application of other known anti-virus and anti-nervous system diseases by administration drugs as described alone, together with other drugs, pharmacologically acceptable salt or prodrug.

8. The drugs, derivatives and analogs according to claims 5, 6 and 7 wherein:
The drugs, derivatives and analogues as described are administered by oral, parenteral, subcutaneous, intravenous, intramuscular, intraperitoneal, transdermal, buccal, intrathecal, intracranial, intranasal or local channels.
